(19) United States
(12) Patent Application Publication
(10) Pub. No.: US 2012/0022358 A1
(43) Pub. Date: Jan. 26, 2012

(54) METHOD AND MAMMOGRAPHY APPARATUS FOR IMAGE-ASSISTED BIOPSY EXTRACTION

(76) Inventor: Daniel Fischer, Erlangen (DE)

(21) Appl. No.: 13/187,703

(22) Filed: Jul. 21, 2011

(30) Foreign Application Priority Data
Jul. 21, 2010 (DE) 10 2010 031 738.1

(51) Int. Cl. A61B 10/02; A61B 6/00

(52) U.S. Cl. 600/407

(57) ABSTRACT

In a device and associated method, images of a biopsy unit in x-ray images for a second volume set are determined and replaced with x-ray image data of corresponding x-ray images for a first volume set, to produce a revised second volume set.

### BACKGROUND OF THE INVENTION

1. Field of the Invention

The present invention concerns methods and mammography systems for implementing a biopsy with imaging assistance.

2. Description of the Prior Art

In a biopsy a tissue sample is extracted—for example from the tissue of the mamma of a patient—for histological examination. An extraction of the tissue sample can take place with the assistance of imaging methods. An overview volume set of the breast is created with the aid of a tomosynthesis method. X-ray images of the breast are acquired to create the overview volume set. For x-ray image acquisition, an x-ray tube is moved (for example in an orbit) over a detector. For example, in the tomosynthesis the x-ray tube moves in an angle range from +25° to −25°. An x-ray radiation in the x-ray source is then triggered at definable intervals, and the respective x-ray image is read out from the detector and buffered. An overview volume set is subsequently created with a reconstruction method. Regions for the extraction of a tissue sample are localized using the slice images of the overview volume set, and from this the coordinates are determined for the entry position, direction and depth of a biopsy needle. The biopsy needle is introduced into the breast for tissue extraction. Since an exact fixing of the breast between the detector and a compression plate is to be maintained with great difficulty over the entire examination duration, and the tissue variation to be removed is often only a few cubic millimeters in size, a control volume set of a mamma with a biopsy needle introduced into the breast tissue is created before the extraction of the tissue sample. The alignment and the position of the biopsy needle tip can be checked by means of evaluation of the slice images of the control volume set, and an adaptation and alignment of the biopsy needle can be conducted. The checking and adaptation of the alignment of the biopsy needle tip using the slice images of the control volume set is hindered by what are known as out-of-plane artifacts.

### SUMMARY OF THE INVENTION

An object of the invention is to provide a device an a method for an image-monitored biopsy extraction without the disadvantage described above.

A device according to the invention has a mammography apparatus with a biopsy unit in which a first volume set without a biopsy needle in the tissue of a breast is acquired and a second volume set with the biopsy needle in the tissue of the breast is acquired.

In x-ray images a marking unit marks portions of the biopsy unit that are imaged to produce the second volume set. By means of an insertion unit, the marked regions in the x-ray images for the second volume set are replaced with x-ray image data of corresponding x-ray images for the first volume set.

The invention has the advantage that out-of-plane artifacts are avoided.

The invention also has the advantage that the biopsy needle is reproduced in the corrected x-ray image data for the second volume set.

The invention has the further advantage that the depiction of the breast tissue is clear and distinct.

### BRIEF DESCRIPTION OF THE DRAWINGS

FIG. 1 is a front view of a biopsy unit for use in accordance with the invention.

FIG. 2 shows a projection curve of an x-ray unit in accordance with the invention.

FIG. 3 is a plan view of the biopsy unit of FIG. 1.

FIG. 4 is a further plan view of the biopsy unit of FIG. 1.

FIG. 5 is a block diagram of an embodiment of the invention.

### DESCRIPTION OF THE PREFERRED EMBODIMENTS

In this device and the associated method, images of a biopsy unit are determined in x-ray images for a second volume set and replaced with x-ray image data of corresponding x-ray images for a first volume set, and a revised second volume set is created.

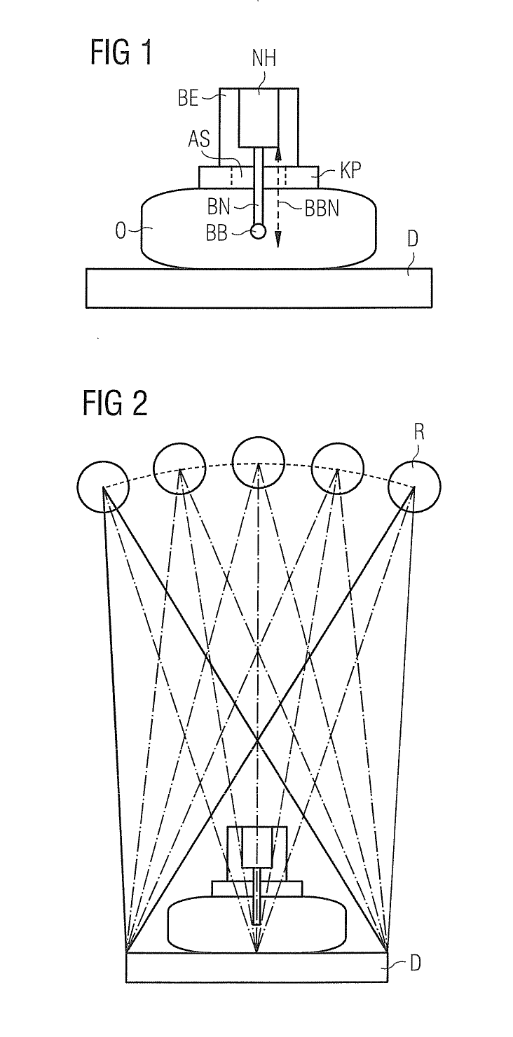

FIG. 1 shows a subject O compressed and fixed between compression plate KP and surface of a detector D. For example, this subject O can be the breast of a patient. The biopsy needle BN is connected via a biopsy needle mount NH with the positioning unit BE. With the positioning unit BE the biopsy needle BN is aligned on a localized tissue variation. For example, this tissue variation can be localized by means of an image evaluation algorithm in slice images from the first volume set (which is also designated as an overview volume set). The biopsy needle BN is navigated to a selected tissue variation and aligned on said selected tissue variation in a computer-controlled manner after a prioritization of the localized tissue variations. For this the biopsy needle BN is aligned within a recess AS of the compression plate KP on the tissue variation. In FIG. 1 the biopsy needle BN is introduced in the tissue of the breast and aligned on a tissue variation. The tip of the biopsy needle is positioned directly in front of the localized tissue variation. The extraction of the tissue sample takes place only after a check as to whether the biopsy needle is correctly aligned.

A trajectory of an x-ray tube R of a mammography apparatus is reproduced in FIG. 2. The trajectory can thereby take place as shown along a circle segment or within a plane. A first sequence—for example of 25 x-ray images—is created for a tomosynthesis. The x-ray tube R is moved across the detector D along a circular arc in an angle range between 25° and −25°. Subjects of different height in the breast are projected differently on the detector at different angles. Conspicuous tissue structures in the mamma are emphasized by suitable filtering, displacement and summation during the subsequent reconstruction, for example via the methods of filtered back projection. The reconstruction leads to a series of slice images in different depth layers parallel to the detector surface. In the described exemplary embodiment, a first sequence ST1 of x-ray images of the compressed breast and a second sequence ST2 of x-ray images are produced before a biopsy extraction from the tissue of the breast. A first volume set OVS—an overview volume set—is created with the first sequence ST1, and a second volume set KVS—a control volume set—is created with the second sequence ST2. It is disadvantageous that, given slice images from the tomosynthesis reconstruction process, arise in the individual x-ray exposures artifacts due to the imaging of biopsy needle, biopsy needle mount or biopsy needle mount with positioning unit. The inventive idea now lies in detecting and extracting images of biopsy needle BN, biopsy needle mount NH and parts of the positioning unit BE in the individual x-ray image data sets or projections of the second sequence, and in replacing these with x-ray image data from x-ray image sets from the first sequence.

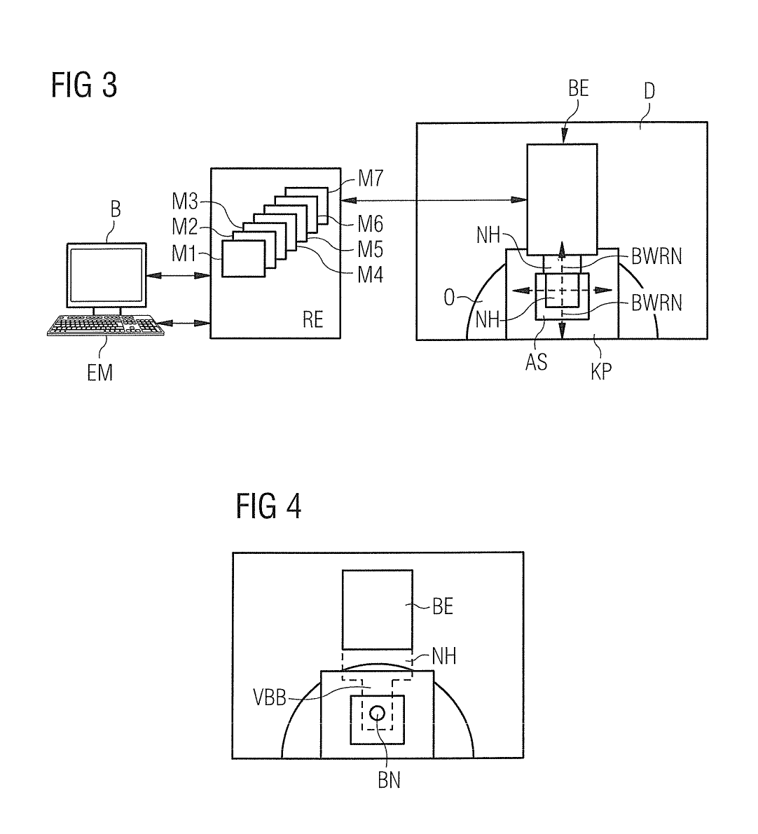

A plan view of the biopsy unit BE, NH is shown in FIG. 3. The creation of the overview volume set OVS, the control volume set KVS, the pre-positioning of the biopsy needle BN, the exact alignment of the biopsy needle and the extraction of the bioptate are implemented by the computer RE connected with the biopsy unit BE.

In this embodiment the individual processing units or, respectively, processing steps listed above are integrated into the modules M1 through M7 of the computer RE. The creation of a first sequence ST1 of x-ray exposures is initiated in the first module Ml. A unit to create an overview volume set (a first volume set OVS) and a readout unit STST1 to readout slice images are integrated into a second module M2. A localizer unit LE, a coordinate determination unit PE for localized tissue variations and a preparation unit AB to output control data for the alignment and insertion of the biopsy needle BN are arranged in the subsequent third module M3. In the fourth module M4 a workflow of a second sequence ST2 of x-ray images is initiated and a control volume set KVS is calculated. A marking unit MA and a determination unit KR to determine corresponding x-ray images in the first and second sequence ST1, ST2 are integrated into the fifth module M5. An insertion unit EET to create corrected x-ray image data is arranged in the sixth module M6.

In this module x-ray image data from the first sequence ST1 are inserted into corresponding x-ray images from the second sequence ST2. The x-ray image parts in the x-ray images of the second sequence ST2 in which biopsy needle BN, biopsy needle mount NH and portions of the positioning unit BE are imaged are replaced by x-ray image data from the first sequence ST1. In the seventh module M7 the calculation of a corrected control volume set BVS is implemented. In the corrected control volume set BVS the biopsy needle BN is then virtually inserted corresponding to its position and alignment.

An input unit means EM and a monitor unit B are connected with the computer RE. Among other things, the biopsy unit consists of a biopsy needle BN, the biopsy needle mount NG and a positioning unit BE for the biopsy needle mount NH. The breast is positioned under the compression plate corresponding to the localization of a tissue variation to be examined, such that the tissue variation is located below the recess AS. For fine adjustment or, respectively, alignment of the biopsy needle this is positioned with the aid of the control signals provided by the computer RE. The desired positioning and the current positioning of the biopsy needle tip are compared by means of the computer RE. The control unit BE controls the biopsy needle BN corresponding to the indicated movement directions BWRN, BBN until the biopsy needle tip is aligned exactly on the tissue variation. For tissue extraction the tip of the biopsy needle is inserted into the tissue of the breast up to a few millimeters in front of the localized tissue variation. For monitoring a second sequence ST2 of x-ray exposures for the tomosynthesis reconstruction process is acquired before the extraction of the tissue.

A plan view of the positioning unit BE for the biopsy needle mount NH and the biopsy needle BW is shown in FIG. 4. The dashed line reproduces possible dimensions of the biopsy needle mount NH. During the x-ray acquisitions, portions of the biopsy needle mount NH and the biopsy needle BN are imaged in these.

The breast is fixed and compressed in the compression unit of the mammography apparatus. The breast remains in this until the biopsy extraction.

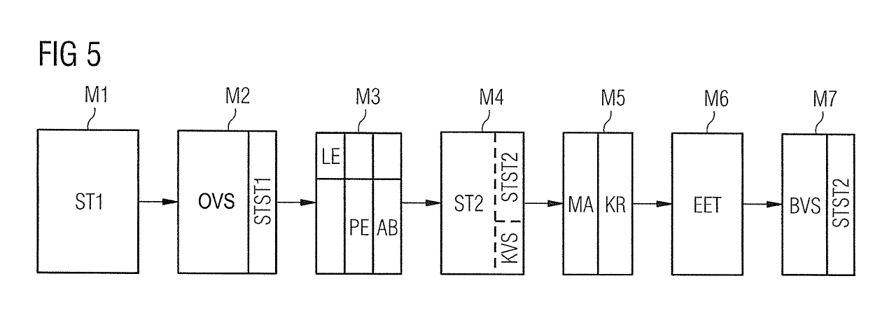

A block diagram for the avoidance of out-of-plane artifacts in a tomosynthesis reconstruction process is depicted in FIG. 5. The respective slices images available for assessment are retrieved from a second, revised volume set BVS. Schematic units or, respectively, processing procedures are summarized in the individual modules M1 through M7. The creation of a first sequence of x-ray exposures or, respectively, projections ST1 for a first tomosynthesis reconstruction process is initiated in the first module M1. The data from these are summarized in a first volume set OVS (which is also designated as an overview volume set) in the second module M2. A number of slice images STST1 can then be retrieved from this. A localizer unit LE, a positioning unit PE and an alignment unit AB are integrated into the third module M3. The localizations of tissue variations in the mamma are determined with this third module M3. The biopsy needle is introduced into the breast up to just before the tissue variation. In the fourth module M4 a second sequence of x-ray exposures ST2 is initiated and implemented for a second tomosynthesis reconstruction process. The position and alignment of the biopsy needle BN introduced into the breast is monitored with these slice images. The regions in the projection images that have been occluded by biopsy needle, needle mount or positioning unit BE are marked in the fifth module M5. The marking can take place with the aid of a segmentation algorithm. The marking of the regions in the respective x-ray images can take place based on the position and shape of the biopsy needle of the needle mount and the geometric conditions of x-ray head and detector unit. The tissue in the breast can be deformed by the introduction of the biopsy needle. In order to keep the computing cost of a registration or mapping method between x-ray images from the first and second sequence ST1, ST2 low, the x-ray image data in the surroundings of the introduced biopsy needle are correspondingly adapted by a deformation algorithm. In a correspondence unit KR, the corresponding x-ray images from the first and second sequence ST1, ST2 are respectively determined in an additional step. This can take place via registering methods or an image mapping method. If the x-ray image pairs are determined, x-ray image data from marked regions of the second sequence ST2 are replaced by corresponding regions from x-ray image data of the first sequence ST1 by means of the insertion unit EET arranged in the sixth module M6. The marked region can also be enlarged by a safety interval. The data from the x-ray images that were created without biopsy needle, biopsy mount and/or biopsy unit can be filtered before they are inserted or, respectively, after they were inserted. The x-ray images that were created with and without biopsy needle can be registered with one another. Projections that were acquired from a different angle can also be used to register the x-ray images. During the registration the displacement of the tissue given an introduced biopsy needle can be taken into account by means of a deformation model. The unmarked regions can be mixed with the projection images without biopsy needle in order to reduce the noise. A revised, second volume set BVS is determined in the seventh module M7. The reconstruction with the revised x-ray image data is then initiated after the marked regions in the x-ray images of the second sequence ST2 have been replaced and a tissue displacement has possibly also been taken into account via a deformation model.

Images of the biopsy needle BN, the needle mount NH and/or parts of the positioning unit BE can be inserted into the slice images after the reconstruction. 3D model data can be accessed to depict the individual articles. However, the contours of the biopsy needle BN can also be reconstructed separately. The position of the region to be biopsied that is established in the pre-exposures is indicated in the slice images.

Instead of tomosynthesis projections, a first stereotactic image pair for localization of the tissue variation and a second stereotactic image pair for position monitoring of the biopsy needle can also be created. Here as well the portions of the biopsy needle that are imaged in the x-ray exposures for the second stereotactic image pair can be replaced with corresponding portions of the x-ray exposures for the first stereotactic image pair.

Although modifications and changes may be suggested by those skilled in the art, it is the intention of the inventor to embody within the patent warranted hereon all changes and modifications as reasonably and properly come within the scope of his contribution to the art.

I claim:

1. A mammography apparatus comprising:
an imaging system adapted to irradiate a subject located in the imaging system;
a biopsy unit adapted to interact with the subject to extract a tissue sample therefrom;
said imaging system being configured to obtain a first volume set of image data without the biopsy unit being present, and to obtain a second volume set of image data with said biopsy unit being present and interacting with the subject;
a computerized processor to which said first and second volume sets are supplied, said computerized processor comprising a marking unit configured to electronically mark portions of the biopsy unit that are present in image data in said second volume set, thereby producing marked regions in said second volume set; and
said processor comprising an insertion unit configured to insert image data, as inserted image data, into said marked regions, said inserted data comprising image data from said first volume set that corresponds to image data in said second volume set contained in said marked regions of said second volume set, and to make said second volume set with said inserted data therein, available at an output of the processor.

2. A mammography apparatus as claimed in claim 1 wherein said processor comprises a determination unit configured to determine respective x-ray images in said first volume set that correspond to respective x-ray images in said second volume set.

3. A mammography apparatus as claimed in claim 1 wherein said biopsy unit comprises a biopsy needle, and wherein said processor comprises a coordinate determination unit configured to identify coordinates in said second volume data set of a localized tissue variation in said second volume set, and to guide said biopsy needle to extract a sample from the subject at said coordinates.

4. A mammography apparatus as claimed in claim 3 wherein said processor comprises a virtual insertion unit configured to generate a volume data set corresponding to said second volume data set in which a current position and shape of said biopsy needle are virtually inserted.

5. A method for operating a mammography apparatus comprising:
with an imaging system, irradiating a subject, located in the imaging system, with x-rays;
providing a biopsy unit adapted to interact with the subject to extract a tissue sample therefrom;
with said imaging system, obtaining a first volume set of image data without the biopsy unit being present, and to obtain a second volume set of image data with said biopsy unit being present and interacting with the subject;
in a computerized processor supplied with said first and second volume sets electronically marking portions of the biopsy unit that are present in image data in said second volume set, thereby producing marked regions in said second volume set; and
in said processor, inserting image data, as inserted image data, into said marked regions, said inserted data comprising image data from said first volume set that corresponds to image data in said second volume set contained in said marked regions of said second volume set, and making the second volume set with the inserted data therein available at an output of the processor.

6. A method as claimed in claim 5 comprising, in said processor, determining respective x-ray images in said first volume set that correspond to respective x-ray images in said second volume set.

7. A method as claimed in claim 5 wherein said biopsy unit comprises a biopsy needle, and comprising, in said processor, identifying coordinates in said second volume data set of a localized tissue variation in said second volume set, and guiding said biopsy needle to extract a sample from the subject at said coordinates.

8. A method as claimed in claim 7 comprising, in said processor, generating a volume data set corresponding to said second volume data set in which a current position and shape of said biopsy needle are virtually inserted.
